(19) United States
(12) Patent Application Publication
(10) Pub. No.: US 2016/0249829 A1
(43) Pub. Date: Sep. 1, 2016

(54) ACTUATED FOOT ORTHOTIC WITH SENSORS

(71) Applicant: The Board of Regents of the Nevada System of Higher Education on Behalf of the Univ. of Nevada, Las Vegas, NV (US)

(72) Inventors: Mohamed Trabia, Las Vegas, NV (US); Janet Dufek, Henderson, NV (US)

(21) Appl. No.: 15/034,598

(22) PCT Filed: Nov. 5, 2014
(86) PCT No.: PCT/US14/64148

§ 371 Date: May 5, 2016

Related U.S. Application Data
(60) Provisional application No. 61/899,960, filed on Nov. 5, 2013.
(60) Provisional application No. 62/017,544, filed on Jun. 26, 2014.

(51) Int. Cl. A61B 5/103; A43B 3/00; A61B 5/00; A43B 17/00

(52) U.S. Cl. CPC A61B 5/1038; A43B 17/00; A43B 3/0005; A61B 5/7246; A61B 5/0077; A61B 5/6807; A61B 5/4836; A61B 5/7275; A61B 2562/0247

(57) ABSTRACT

Systems and methods of developing a tissue deformation profile for a foot of a patient. The tissue deformation profile can be used to identify a desired configuration for an orthotic device. A method uses a sensing orthotic device to assist in the treatment of foot issues. The orthotic device can have at least two sensors and at least one actuator for adjusting physical parameters of the orthotic device.

### CROSS-REFERENCE TO RELATED APPLICATIONS

This application claims priority to U.S. Provisional Patent Application No. 61/899,960, filed Nov. 5, 2013, and to U.S. Provisional Patent Application No. 62/017,544, filed Jun. 26, 2014, each of which is hereby incorporated herein by reference in its entirety.

### BACKGROUND

1. Field

The present invention relates to the field of foot orthotics. More particularly, the present invention relates to a tissue deformation model that can be used to develop a foot orthotic. The present invention also relates to the use of sensors in a foot orthotic and the presence of actuators associated with the orthotic that can cause alteration in the shape, performance and position of the orthotic by internal (embedded) or external (remote, wired or wireless) processor control.

2. Background of the Art

The human foot functions in a unique way to provide the body with balance between support and mobility in order to optimize human locomotion. Healthy individuals manage very efficiently the plantar pressure distribution on the foot by the combination of bones, joints, muscles, and ligaments acting in concert with neurological input. However, it has been observed that diabetic patients apply pressure to various footpad regions unevenly, which can lead to foot ulcerations. These ulcerations are difficult to detect since diabetic patients often experience sensory neuropathy (lack of sensation), leading to excessive pressure along the infected footpad areas. These elevated pressure levels can lead to uncontrollable inflammation, focal tissue ischemia, necrosis, and skin ulceration; ultimately, this can result in multiple complications, including greater rates of infection and eventual amputation of infected tissues. In addition to the reduction in quality of life for affected patients, foot ulceration escalates the medical cost of treating these patients over extended periods. Thus, there is a need in the art for systems and methods of modeling the pressure distribution and tissue deformation within a foot of an individual. There is a further need in the art for orthotic devices that are produced in accordance with the modeled pressure distribution and tissue deformation of the foot of an individual. There is still a further need in the art for orthotic devices that are (1) capable of measuring various characteristics of the foot of an individual during movement of the individual and (2) selectively adjustable in response to the measured characteristics of the foot to optimize performance of the orthotic device.

Orthotics on bodies, particularly to support joints, and also specifically in footwear have been found to provide significant reparative and training benefits to user patients that suffer from various foot injuries or conditions. The available orthotics are broadly classified as passive orthotics and active orthotics. Passive orthotics are basically with standard component shapes and manually adjustable fittings (braces, clamps, straps, sliding pads and the like), or may be molded to specifically fit individuals. Some may be purchased without need of medical assistance or supervision, such as foot sole orthotics sold commercially in sport and retail stores.

Active orthotics are more alterable after placement and/or may contain sensing devices that relay useful information to trained personnel to assist in modification of the prosthetic on an individual user or to provide information about the quality of progress in the use of the orthotics. Some orthotics are used purely for training purposes (both medical and sports training) to provide feedback on the performance of the individual using the orthotic device.

Examples of disclosed active orthotic devices and systems enabling their performance are found, for example, in U.S. Patent Application Publication No. 2012/0238914 to Goldfield, showing an actively controlled orthotic device that includes active components that dynamically change the structural characteristics of the orthotic device according to the orientation and locomotion of the corresponding body part, or according to the changing needs of the subject over a period of use. Accordingly, the orthotic device can be effectively employed to provide locomotion assistance, gait rehabilitation, and gait training. Similarly, the orthotic device may be applied to the wrist, elbow, torso, or any other body part. The active components may be actuated to effectively transmit force to a body part, such as a limb, to assist with movement when desired. Additionally or alternatively, the active components may also be actuated to provide support of varying rigidity for the corresponding body part.

U.S. Patent Application Publication No. 2013/0150980 to Swift discloses a powered lower extremity orthotic, including a shank link coupled to an artificial foot, a knee mechanism connected to the shank link and a thigh link, is controlled by signals from various orthotic mounted sensors such that the artificial foot follows a predetermined trajectory defined by at least one Cartesian coordinate.

U.S. Patent Application Publication No. 2011/0106274 to Ragnarsdottir disclosed a system and method associated with the movement of a limb. In one example, the system, such as a prosthetic or orthotic system, includes an actuator that actively controls, or adjusts, the angle between a foot unit and a lower limb member. A processing module may control movement of the actuator based on data obtained from a sensor module. For instance, sensing module data may include information relating to the gait of a user and may be used to adjust the foot unit to substantially mimic the movement of a natural, healthy ankle. The system may further accommodate, for example, level ground walking, traveling up/down stairs, traveling up/down sloped surfaces, and various other user movements. In addition, the processing module may receive user input or display output signals through an external interface. For example, the processing module may receive heel height input from the user.

U.S. Patent Application Publication No. 2010/0179668 to Herr describes a hybrid terrain-adaptive lower-extremity apparatus and methods that perform in a variety of different situations by detecting the terrain that is being traversed, and adapt to the detected terrain. In some embodiments, the ability to control the apparatus for each of these situations builds upon five basic capabilities: (1) determining the activity being performed; (2) dynamically controlling the characteristics of the apparatus based on the activity that is being performed; (3) dynamically driving the apparatus based on the activity that is being performed; (4) determining terrain texture irregularities (e.g., how sticky is the terrain, how slippery is the terrain, is the terrain coarse or smooth, does the terrain have any obstructions, such as rocks) and (5) a mechanical design of the apparatus that can respond to the dynamic control and dynamic drive.

U.S. Patent Application Publication No. 2009/0171469 to Thorsteinsson describes an orthotic frame that has proximal and distal frame members joined by a knee joint, and a foot support joined by an ankle joint to a distal end of the distal frame. A knee actuator connected between the proximal and distal frame members has a selective stiffness allowing selection of a relatively rigid stiffness during stance and a relatively flexible stiffness during swing. The stiffness of the knee actuator is selected according to the gait cycle, either mechanically according to dorsi flexion of the ankle joint or electronically according to gait cycle phases recognized based on read sensor data. An ambulatory unit gathers data from sensors located on the orthotic frame. Sensor data may be provided to a base unit for diagnostic and biomechanical evaluation, or evaluated by the ambulatory unit to control active components of the orthotic frame according to the recognized gait cycle phases for functional compensation.

U.S. Patent Application Publication No. 2013/0079693 to Ranky describes a method of constructing a sensor includes depositing a first material in a predetermined arrangement to form a structure. The depositing results in at least one void occurring within the structure. The method further includes depositing a second material within the voids. The second material may have electrical properties that vary according to deformation of the second material. The method also includes providing electrical access to the second material to enable observation of the one or more electrical properties. A sensor includes a structure that has one or more voids distributed within the structure. The sensor also includes a material deposited within the one or more voids. The material may be characterized by one or more electrical properties such as piezoresistivity. The sensor includes a first contact electrically coupled to a first location on the material, and a second contact electrically coupled to a second location on the material.

U.S. Patent Application Publication No. 2010/0211355 to Horst describes a foot pad device and a method of obtaining weight data from a force sensor in a foot pad worn by a user engaging in a footstep, including placing the force sensor under the ball of the foot of the user and/or the heel of the foot of the user; receiving an entered patient weight value for the user; collecting force data from the force sensor; calculating a weight value based on the collected force data and a scaling and/or offset parameter; comparing the calculated weight value to the entered patient weight value; comparing the calculated weight value to zero; adjusting the scaling and/or offset parameter; and repeating the steps periodically. The method may include comparing the collected force data to a functionality indication range, flagging the force sensor if the collected force data is outside the functionality indication range, and disregarding force data from the flagged force sensor.

U.S. Patent Application Publication No. 2010/0274447 to Stumpf describes a transducer system and includes a plurality of transducer elements formed on a flexible substrate with localized circuit elements and interconnects associated with each transducer element, which when embedded in a carrier layer may also be used as flexible sensory “smart skin” for a prosthesis or robotic element such as a cybernetic hand permitting imaging of force distributions for robotic gripping applications. In another example, a prosthesis may include transducer matrix film as part of its external surfaces for providing a simulated sensor of touch or contact. For example, an artificial foot may be covered by transducer matrix film providing sensory data about gait, foot pressure and other information to affect or improve rehabilitation.

U.S. Patent Application Publication No. 2012/0095377 to Smith describes a multi-fit orthotic structure including an attachment system for coupling the orthotic structure to a wide variety of subjects without requiring a custom fit. In one embodiment, active mobility assistance is provided via an orthotic system capable of integrating a linear actuator and linkage system to deliver torque to the lower leg of a subject to facilitate flexion and/or extension motion of the subject's leg. The orthotic structure is attached to the subject using a textile suspension system which does not require the orthotic structure to interface directly in the knee region or at the lateral areas of the thigh and calf of the subject, thus providing an ideal fit for the widest possible range of subjects with the minimum number of required sizes.

These references and devices each have their unique attributes and functions. All references cited herein are incorporated in their entirety. Inclusion of materials, components, software and processing technology in some of these references can be converted to use in the practices of the novel methods and composite structures disclosed herein.

### SUMMARY

A system is provided for modeling tissue deformation of a foot of a patient as the patient walks relative to an axis of movement. The system can include means for measuring pressure applied by the foot of the patient as the patient walks relative to the axis of movement. In one optional aspect, the means for measuring pressure can include at least one pressure sensor. In another optional aspect, the means for measuring pressure can further include a pressure-sensitive device positioned such that the axis of movement passes through the pressure-sensitive device. The pressure-sensitive device can have a transverse axis substantially perpendicular to the axis of movement and a contact surface, and the at least one pressure sensor can be configured to produce at least one pressure output indicative of a pressure applied to the contact surface as the patient walks over the pressure-sensitive device relative to the axis of movement. Optionally, the pressure-sensitive device can be a pressure-sensitive mat or a pressure-sensitive insole. The system can also include at least one camera. Optionally, the at least one camera can include a plurality of cameras, such as, for example, first and second cameras. Each of the first and second cameras can be configured to produce one or more images of the foot of the patient as the patient walks relative to the axis of movement. The first camera can have an orientation axis positioned substantially perpendicular to the axis of movement. The second camera can have an orientation axis positioned substantially parallel to the axis of movement. The system can further include a processor in operative communication with the at least one pressure sensor and the first and second cameras. The processor can be configured to receive the at least one pressure output from the at least one pressure sensor. The processor can be configured to receive the one or more images produced by the first and second cameras. The processor can be configured to determine deformation of the foot of the patient based upon the one or more images produced by the first and second cameras. The processor can be further configured to correlate the at least one pressure output with a corresponding deformation of the tissue of the foot of the patient to thereby produce a tissue deformation profile. Optionally, the system can include a three-dimensional scanner configured to produce a three-dimensional image of the foot of the patient. The processor can be positioned in operative communication with the three-dimensional scanner and configured to receive the three-dimensional image of the foot of the patient.

A method is provided for developing a tissue deformation profile for a foot of a patient. The method can include producing a plurality of pressure outputs indicative of the pressure applied by the foot during walking. The method can also include producing one or more images depicting the compression of the foot during walking. The method can further include determining, using a processor, the deformation of the foot based upon the one or more images depicting the compression of the foot. The method can still further include correlating, using a processor, the pressure applied by the foot and the deformation of the foot to produce the tissue deformation profile.

A method of producing an orthotic device is provided. The method can include producing a tissue deformation profile and using the tissue deformation profile to select a desired configuration of the orthotic device. The method can further include producing the orthotic device in accordance with the desired configuration.

A system and method are provided for in situ sensitometric evaluation of an orthotic in footwear of a patient and the in situ adjustment of the orthotic using actuators associated with the orthotic in the footwear. At least two different types of sensors are distributed along various regions of the footpad of a patient (e.g., heel, metatarsal and toes). The at least two sensors should include pressure sensors to measure pressure applied to specific areas within the footpad during the gait of a patient and displacement sensors which measure deformation in the footpad in the various regions during application of gait pressure.

Additional advantages of the invention will be set forth in part in the description which follows, and in part will be obvious from the description, or may be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### BRIEF DESCRIPTION OF THE FIGURES

FIGS. 1A-1C show a sequence of images obtained during the development of a tissue deformation model as disclosed herein.

FIGS. 2A-2B show images obtained from different perspectives during the development of a tissue deformation model as disclosed herein.

FIG. 3 shows a vertical view of an orthotic for placement within a shoe, the orthotic having sensors and actuators distributed therein.

FIG. 4 shows a table indicating steps used in a general method for adjusting the physical properties of an orthotic as disclosed herein.

FIG. 5 shows an experimental flow chart illustrating progression of data capture and reduction, leading to the desired footpad pressure values.

FIG. 6 is a schematic diagram depicting a system for modeling tissue deformation as disclosed herein.

FIG. 7 is a top view of an exemplary system for modeling tissue deformation as disclosed herein.

FIG. 8 is a side view of a pressure-sensitive device of an exemplary system for modeling tissue deformation as disclosed herein.

### DETAILED DESCRIPTION

The present invention can be understood more readily by reference to the following detailed description, examples, drawings, and claims, and their previous and following description. However, before the present devices, systems, and/or methods are disclosed and described, it is to be understood that this invention is not limited to the specific devices, systems, and/or methods disclosed unless otherwise specified, as such can, of course, vary. It is also to be understood that the terminology used herein is for the purpose of describing particular aspects only and is not intended to be limiting.

The following description of the invention is provided as an enabling teaching of the invention in its best, currently known embodiment. To this end, those skilled in the relevant art will recognize and appreciate that many changes can be made to the various aspects of the invention described herein, while still obtaining the beneficial results of the present invention. It will also be apparent that some of the desired benefits of the present invention can be obtained by selecting some of the features of the present invention without utilizing other features. Accordingly, those who work in the art will recognize that many modifications and adaptations to the present invention are possible and can even be desirable in certain circumstances and are a part of the present invention. Thus, the following description is provided as illustrative of the principles of the present invention and not in limitation thereof.

As used throughout, the singular forms “a,” “an” and “the” include plural referents unless the context clearly dictates otherwise. Thus, for example, reference to “a pressure-sensitive device” can include two or more such pressure-sensitive devices unless the context indicates otherwise.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

As used herein, the terms “optional” or “optionally” mean that the subsequently described event or circumstance may or may not occur, and that the description includes instances where said event or circumstance occurs and instances where it does not.

The word “or” as used herein means any one member of a particular list and also includes any combination of members of that list.

As used herein, the term “processor” refers to any conventional processing element or logic that is capable of performing the functions disclosed herein. In exemplary aspects, the processor can be provided as part of a computing device, such as, for example and without limitation, a personal computer. However, it is understood that remote computing devices (including smart phones, tablets, and the like) can be used. In other exemplary aspects, the processor can be positioned in operative communication with a memory that stores software and other modules sufficient to allow the processor to function as disclosed herein.

### A System for Modeling Tissue Deformation

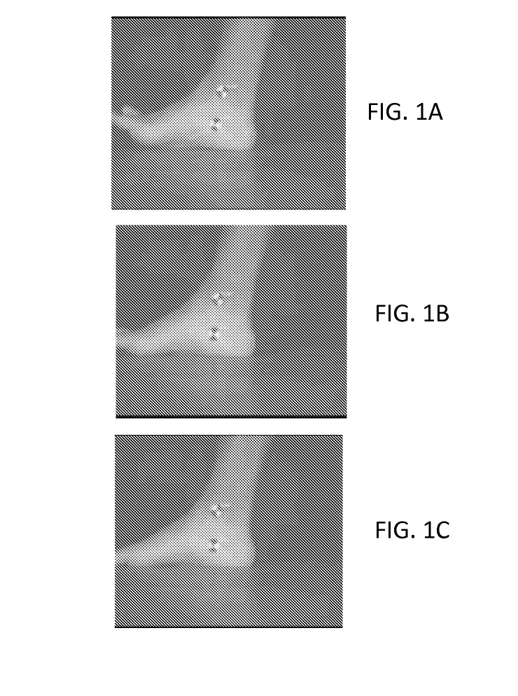

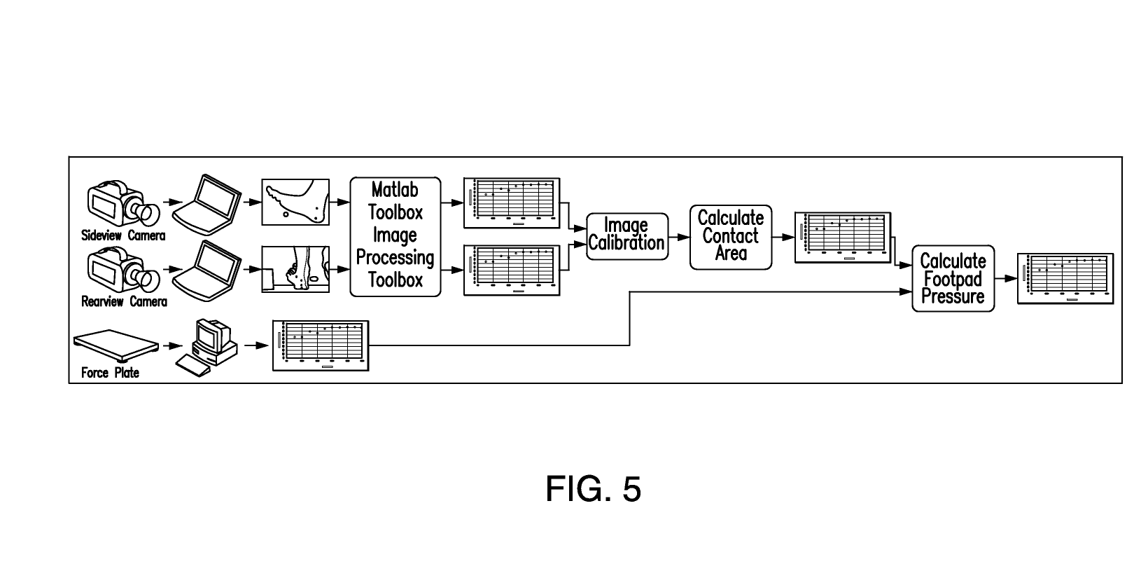

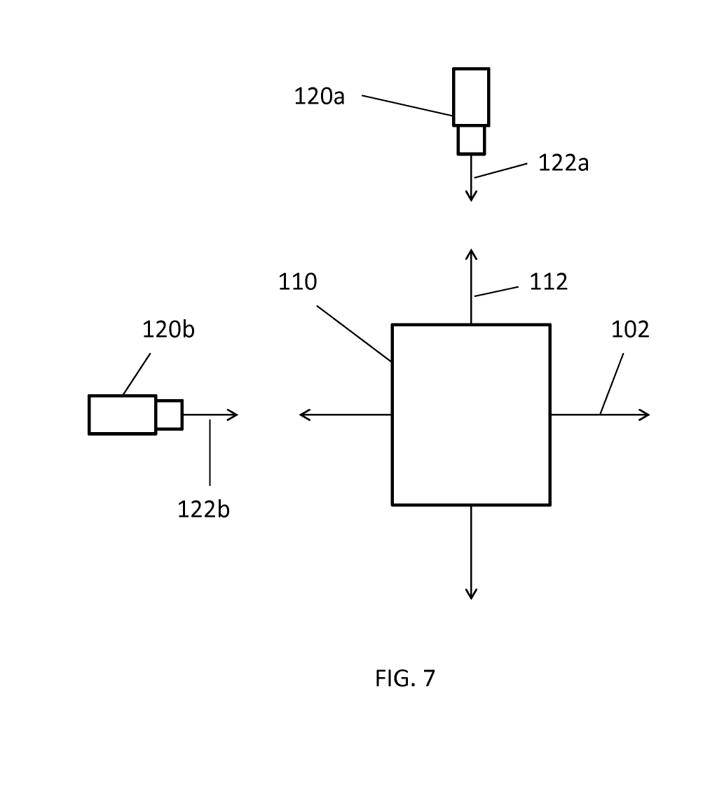

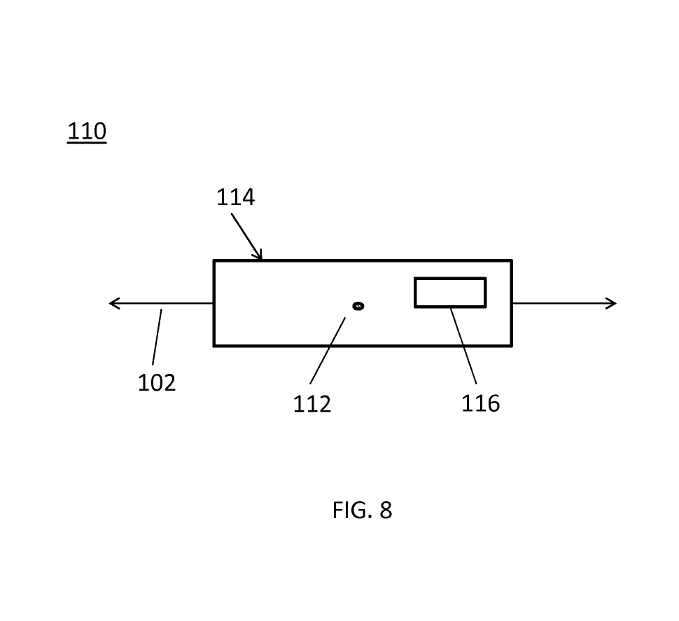

In exemplary aspects, and with reference to FIGS. 1A-2B and 5-8, a is provided for modeling tissue deformation of a foot of a patient. In these aspects, the system 100 can be configured to model the tissue deformation of the foot as the patient walks relative to an axis of movement 102. Weight and other physical characteristics of the patient can be used by the system 100 to optimize modeling of the tissue deformation.

In one aspect, the tissue deformation modeling system 100 can comprise means for producing at least one pressure output indicative of a pressure applied by the foot of the patient as the patient walks relative to the axis of movement 102. In this aspect, the means for producing the at least one pressure output can comprise at least one pressure sensor 116.

In another aspect, the tissue deformation modeling system 100 can comprise means for producing one or more images of the foot of the patient as the patient walks relative to the axis of movement 102. In this aspect, the means for producing one or more images of the foot can comprise at least one camera. In exemplary aspects, the at least one camera comprises at least first and second cameras 120a, 120b. In these aspects, the first camera 120a can have an orientation axis 122a positioned substantially perpendicular to the axis of movement 102, and the second camera 120b can have an orientation axis 122b positioned substantially parallel to the axis of movement. However it is contemplated that any orientation of the cameras 120a, 120b that permits imaging of the foot of the patient can be used. It is further contemplated that any number of cameras can be used to image the foot of the patient.

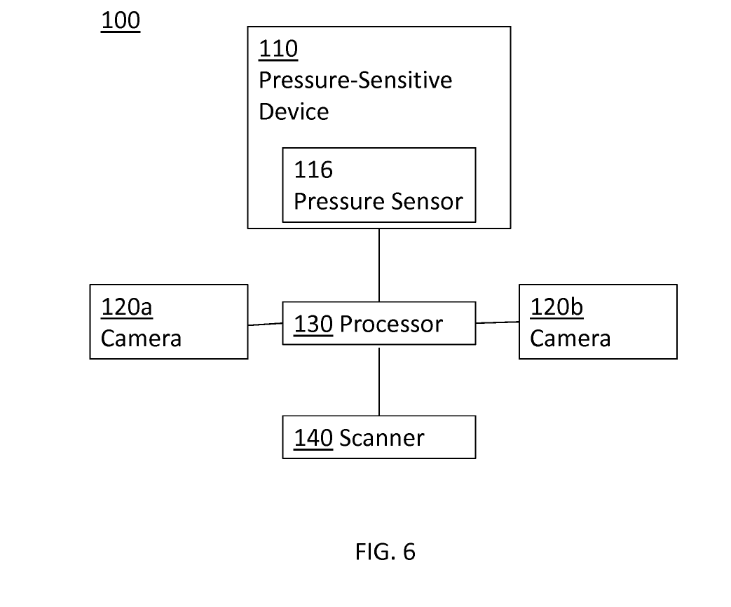

In a further aspect, and with reference to FIG. 6, the tissue deformation modeling system 100 can comprise a processor 130 configured to receive the at least one pressure output and the one or more images. In operation, it is contemplated that the processor 130 can be configured to determine deformation of the foot of the patient based upon the one or more images. It is further contemplated that the processor 130 can be further configured to correlate the at least one pressure output with a corresponding deformation of the tissue of the foot of the patient to thereby produce a tissue deformation profile.

Optionally, in another aspect, the tissue deformation modeling system 100 can further comprise a three-dimensional 140 scanner configured to produce a three-dimensional image of the foot of the patient. In this aspect, and as shown in FIG. 6, the processor 130 can be positioned in operative communication with the three-dimensional scanner 140 and configured to receive the three-dimensional image of the foot of the patient.

A Modeling System with a Pressure-Sensitive Device

Optionally, in exemplary aspects, and as shown in FIGS. 6-8, the tissue deformation modeling system 100 can comprise a pressure-sensitive device 110 positioned such that the axis of movement 102 passes through the pressure-sensitive device. In this aspect, the pressure-sensitive device 110 can have a transverse axis 112 substantially perpendicular to the axis of movement 102, a contact surface 114, and at least one pressure sensor 116 configured to produce at least one pressure output indicative of a pressure applied to the contact surface as the patient walks over the pressure-sensitive device relative to the axis of movement. In exemplary aspects, the pressure-sensitive device 110 can be a pressure-sensitive mat. Alternatively, in other exemplary aspects, it is contemplated that the pressure-sensitive device 110 can be a pressure-sensitive insole. However, it is contemplated that any suitable pressure-sensitive construct can be used.

In another aspect, the tissue deformation modeling system can comprise at least first and second cameras 120a, 120b. In this aspect, each of the first and second cameras 120a, 120b can be configured to produce one or more images of the foot of the patient as the patient walks over the pressure-sensitive device 110 relative to the axis of movement 102. In operation, it is contemplated that the first camera 120a can have an orientation axis 122a positioned substantially parallel to (or, optionally, in alignment with) the transverse axis 112 of the pressure-sensitive device 110 (and substantially perpendicular to the axis of movement 102), and the second camera 120b can have an orientation axis 122b positioned substantially parallel to (or, optionally, in alignment with) the axis of movement.

In a further aspect, and as shown in FIG. 6, the tissue deformation modeling system 100 can comprise a processor 130 in operative communication with the at least one pressure sensor 116 of the pressure-sensitive device 110 and the first and second cameras 120a, 120b. In this aspect, the processor 130 can be configured to receive the at least one pressure output from the at least one pressure sensor 116 of the pressure-sensitive device 110. The processor 130 can be further configured to receive the one or more images produced by the first and second cameras 120a, 120b. In operation, the processor 130 can be configured to determine deformation of the foot of the patient based upon the one or more images produced by the first and second cameras 120a, 120b. The processor 130 can be further configured to correlate the at least one pressure output with a corresponding deformation of the tissue of the foot of the patient to thereby produce a tissue deformation profile.

Optionally, in another aspect, the tissue deformation modeling system 100 can further comprise a three-dimensional scanner 140 configured to produce a three-dimensional image of the foot of the patient. In this aspect, the processor 130 can be positioned in operative communication with the three-dimensional scanner 140 and configured to receive the three-dimensional image of the foot of the patient.

In exemplary aspects, it is contemplated that the processor 130 can be provided as part of a computing device as is known in the art, such as, for example and without limitation, a computer as is known in the art. In these aspects, it is contemplated that the processor 130 can be positioned in operative communication with a memory of a computing device in a conventional manner. In exemplary aspects, it is contemplated that the memory can be configured to receive and store the tissue deformation profile produced by the system 100. In further exemplary aspects, the memory can store software and/or program modules that are configured to analyze the at least one pressure signal and the one or more images to correlate the pressure signals and images with a corresponding tissue deformation. Thus, in operation, it is contemplated that the processor 130 can communicate with the memory to as needed to produce the tissue deformation profile. In further exemplary aspects, it is contemplated that the computing device can comprise a user input positioned in communication with the processor 130 to permit user control of the operations of the system 100. In still further exemplary aspects, the processor 130 can be configured to control the operation of the first and second cameras 120a, 120b. In still further exemplary aspects, the processor 130 can be configured to control the operation of the scanner 140. For example, it is contemplated that the processor can be configured to coordinate and effect the activation of the cameras 120a, 120b and/or scanner 140 to collect images of the foot of the patient at a desired time and/or location.

Methods of Developing a Tissue Deformation Profile and Producing an Orthotic Device Using the Tissue Deformation Profile

Disclosed herein, in one exemplary aspect, is a method of developing a tissue deformation profile for a foot of a patient. In one aspect, the method can comprise producing a plurality of pressure outputs indicative of the pressure applied by the foot during walking. Optionally, in this aspect, the plurality of pressure outputs can be produced using a system as disclosed herein. In another aspect, the method can comprise producing one or more images depicting the compression of the foot during walking. Optionally, in this aspect, the one or more images can be obtained using a system as disclosed herein. In an additional aspect, the method can comprise determining, using a processor, the deformation of the foot based upon the one or more images depicting the compression of the foot. In a further aspect, the method can comprise correlating, using a processor, the pressure applied by the foot and the deformation of the foot to produce the tissue deformation profile. One exemplary protocol for developing a tissue deformation profile is disclosed below in the section labeled, “Development of an Exemplary Tissue Deformation Profile.” However, it is contemplated that other protocols can be used.

In another exemplary aspect, a method of producing an orthotic device is disclosed. In one aspect, the method can comprise producing a tissue deformation profile as disclosed herein. Optionally, in this aspect, the tissue deformation profile can be produced using a system as disclosed herein. In an additional aspect, the method can comprise using the tissue deformation profile to select a desired configuration of the orthotic device. In a further aspect, the method can comprise producing the orthotic device in accordance with the desired configuration. Optionally in this aspect, the orthotic device can be an actuated orthotic device as further disclosed herein, and the step of producing the orthotic device in accordance with the desired configuration can comprise modifying the physical characteristics of the actuated orthotic device to achieve the desired configuration.

In an additional exemplary aspect, a method of producing an orthotic device is disclosed. In one aspect, the method can comprise producing a plurality of pressure outputs indicative of the pressure applied by the foot during walking. Optionally, in this aspect, the plurality of pressure outputs can be produced using a system as disclosed herein. In another aspect, the method can comprise producing one or more images depicting the compression of the foot during walking. Optionally, in this aspect, the one or more images can be produced using a system as disclosed herein. In a further aspect, the method can comprise determining, using a processor, the deformation of the foot based upon the one or more images depicting the compression of the foot. In an additional aspect, the method can comprise correlating, using a processor, the pressure applied by the foot and the deformation of the foot to produce a tissue deformation profile. In yet another aspect, the method can comprise using the tissue deformation profile to select a desired configuration of the orthotic device. In still another aspect, the method can comprise producing the orthotic device in accordance with the desired configuration. Optionally in this aspect, the orthotic device can be an actuated orthotic device as further disclosed herein.

### Development of a Tissue Deformation Model

Ulceration of the foot due to excessive pressure, often leading to amputation, is common among the rapidly increasing diabetic population. A model of tissue deformation of the diabetic foot during locomotion, produced using engineering techniques, can be used to develop a smart orthotic to provide feedback to the diabetic patient when plantar pressure exceeds a minimum criteria. In exemplary aspects, a tissue deformation modeling system as disclosed herein can be used to develop the tissue deformation model. Although disclosed below with reference to particular sample sizes and particular instrument sampling rates, it is contemplated that any suitable sample size and/or instrument sampling rate can be used.

As further disclosed below, tissue deformation models can be developed for both the healthy foot and the diabetic foot. The diabetic foot model can be different than the healthy foot model, and monitoring pressure and the corresponding tissue deformation of the diabetic foot in various regions of the footpad can predict the onset of foot ulcerations.

In exemplary applications, a tissue deformation model of a healthy foot during locomotion and a tissue deformation model of a diabetic foot during locomotion can be produced following a similar protocol. Specifically, the relationship between applied forces and resulting pressure values at various plantar surface areas of the foot and corresponding tissue deformation can be established. Between 20 and 30 adults with Diabetes Mellitus and between 20-30 age-matched and apparently healthy adults, with no diagnosis of diabetes can be used. Participants walk barefoot at their preferred pace, contacting a pressure sensitive mat, pressure-sensitive insole, or other pressure-sensitive device (100 Hz or other suitable frequency) while synchronously obtaining high-speed video data (1000 Hz or other suitable frequency) during the stance phase of gait. Peak pressure values can be identified that are associated with phases of gait, including heel strike and push off. Deformation of the heel pad and metatarsal heads, as viewed sagittally, can be measured using high-speed videography. Conventional system identification techniques, such as, for example and without limitation, software (e.g., Matlab and any other suitable software), can use force-tissue deformation data as the input to construct dynamic models of the various regions of the plantar surface during support. Additionally, a finite element model may be constructed for the regions of the plantar surface to create material models of the tissue that match the experimental data. The results of these two approaches can be compared to determine stiffness and damping of the various regions of the footpad. Uniqueness of the diabetic foot model can be achieved through qualitative comparison of the optimal models of the foot for patients with Diabetes Mellitus and for healthy adults. This descriptive information can be used to show that diabetic foot models exhibit compressive characteristics that are unique to the diabetic population.

The resulting tissue deformation model of the diabetic foot can be used in the development of smart insole sensors and a smart orthotic as further disclosed herein. Smart insole sensors can combine pressure and deformation sensors to provide a low-cost option for continuously monitoring diabetic patients and alert treating physicians in the case of stiffness change of any of the footpad areas. The smart orthotic can incorporate smart materials and actuators to redistribute pressure on the foot, with the goal of reducing the possibility of developing foot ulcerations. In addition, a cell phone “app” can be developed that alerts the individual's physician of situations when increased plantar pressures are being sensed by the orthotic.

Foot ulcerations occur with higher frequency and intensity in the diabetic population. Many factors cause these ulcerations, such as atherosclerotic peripheral arterial disease and peripheral neuropathy. Typically, diabetes is associated with the bonding of proteins and sugar molecules (glycation), which increases the stiffness of ligaments. Coupled with neuropathy, which causes a loss of protective sensation, diabetic patients apply pressure unevenly to various footpad regions, which can lead to foot ulcerations. These elevated pressure levels can lead to uncontrollable inflammation, focal tissue ischemia, necrosis, and skin ulceration; ultimately, this results in multiple complications, including greater rates of infection and the eventual amputation of infected tissues.

Diabetic foot ulcerations are responsible for more hospitalizations than any other complication of diabetes. Additionally, diabetes is the leading cause of non-traumatic lower extremity amputations in the United States. Diabetic foot lesions are responsible for more hospitalizations than any other complication of diabetes. Diabetes is the leading cause of non-traumatic lower extremity amputations in the United States; approximately 5% of diabetics develop foot ulcers each year and 1% of diabetics require amputation. These ulcerations are difficult to detect early. However, early prediction can improve the quality of life for diabetic patients and reduce the overall costs associated with treating these injuries over extended periods.

Several researchers conducted clinical investigations to determine the variations in plantar pressures and stresses in diabetic patients. The researchers compared the plantar tissue stress level in the feet of patients with Diabetes Mellitus, with and without a history of foot ulcers. A control group of healthy, age-matched, and BMI-matched individuals was used. This study found significantly decreased activity and higher stress levels in the diabetic patients with foot ulcers as compared to both diabetic patients without foot ulcers and the control group. This research indicates that diabetic patients with foot ulcers are at a higher risk of plantar tissue injury even at relatively low levels of cumulative tissue stress. Additionally, forefoot structures that influenced forefoot plantar pressures during walking were studied. This research involved use of a foot-pressure mapping system to obtain plantar pressure data of 20 diabetic patients with Peripheral Neuropathy compared to a 20-subject control group. This study measured the variance in plantar peak pressure predicted by forefoot structures in the two groups. In diabetic patients with Peripheral Neuropathy, the metatarsal phalangeal joint angle was determined to be the primary structural variable that predicted plantar peak pressure during walking.

Measuring the plantar pressures of healthy and diabetic subjects is an essential step in determining the response of tissues during gait. Since the footpad consists of skin and fat pads, it is imperative to obtain mechanical properties of both structures by means of a numerical estimation in order to determine how the skin will break down under varying plantar pressures. One study presented the development of a finite element model of the heel at heel strike in order to resolve the variation in heel stiffness. Subject-specific models were developed, based on individual medical images, and compared to experimental plantar pressure measurements from each subject. The results show that the skin layer has greater tissue stiffness than the fat pad. A finite element model of the heel pad was developed, based on magnetic resonance imaging (MRI) of the foot and material testing based on heel indentation.

Since experimental data from diabetic patients can provide insight on where the plantar pressures are applied and their effect on gait and foot ulcerations, some researchers have developed monitoring systems to observe plantar pressure distribution. It was demonstrated that the force sensors, placed in different locations of a shoe insole, could provide a complete output of the force history during gait. To determine the plantar stresses during walking, a load monitoring system was created by the integration of the Hertz contact theory and a biomechanical model. The stress monitor was used to determine the internal stress distribution in the plantar pad for healthy and diabetic subjects. One study demonstrated that mitigating higher plantar pressure by reshaping a shoe insert would manipulate the pressure distribution on the foot.

In exemplary aspects, the methods disclosed herein can be used to develop and compare tissue deformation models for both the diabetic and non-diabetic foot. Monitoring pressure and the corresponding tissue deformation in various regions of the footpad can predict the onset of foot ulcerations. The relationship between the applied forces at various plantar surface areas and corresponding tissue deformation can be established. The models disclosed herein can be used by doctors to monitor a diabetic patient and identify the onset of foot ulcerations during regular checkups before they become visible. This can lead to an improvement of the rates of early treatment of diabetic ulcerations.

The disclosed models can be used to develop a smart orthotic (such as those further disclosed herein) that incorporates actuators to redistribute pressure on the foot, thus reducing the possibility of developing foot ulcerations in diabetic patients. Since the space that can be allocated for the smart orthotic is extremely limited, this device is especially challenging to develop. The orthotic can use multiple sensors to measure pressure at the heel pad, metatarsal heads, and toes. Smart materials, such as piezoelectric bimorph actuators, can be integrated in the orthotic to modulate the pressure on the various regions of the foot.

### An Exemplary Tissue Deformation Model

One exemplary, non-limiting method of producing a tissue deformation model of a foot of a patient is summarized below.

Materials and Methods

a. Participants

Between twenty and thirty adults with Diabetes Mellitus and between 20 and 30 apparently healthy age-matched adults can be used.

b. Instruments

A 0.44×0.37 m pressure-sensitive mat (MatScan, Tekscan Inc.; 100 Hz) can be placed in the middle of a 20-m walkway to measure plantar pressure of the foot. Two high-speed video cameras (Phantom 4.3; 1,000 Hz) can be used to capture foot compression during the support phase of walking. One camera can be placed perpendicular to the pressure mat, at floor level, while a second camera can be placed along the line of progression, focusing on the heel of the foot. A three-dimensional scanner (Polhemus FastSCAN Scorpion) can be used to image the foot in both weight bearing and non-weight bearing conditions for the finite element model development. A typical scale can be used to measure the weight of the participants.

c. Procedures

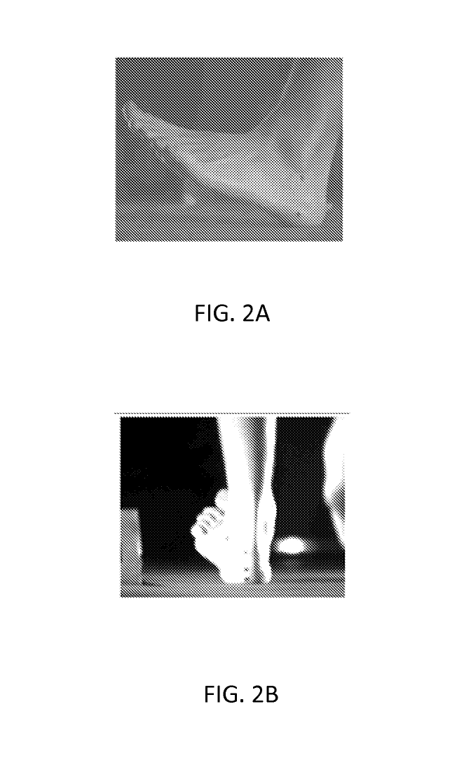

Upon arrival at the laboratory, demographic information can be recorded, including age, height, weight, gender, length of time since the diagnosis of diabetes, and a brief summary of foot problems, if they have occurred. Next, each participant can stand quietly on the mat, barefoot, for the purpose of obtaining a static load-bearing pressure profile. At that point, the participants can practice walking through the 20-m walkway area, contacting the mat in a natural fashion with their right foot. When comfortable with this task, participants can walk through the 20-m walkway 3-5 times, contacting the pressure mat with their right foot; simultaneously, ground reaction force and/or high-speed video of foot compression images can be recorded (FIGS. 1A-2B). Video records, force, and/or pressure data can be synchronized by means of an external trigger on the high-speed video cameras. Following completion of a minimum of three successful steps (in the field of view of the cameras), a three-dimensional scan of the participant's foot can be completed, using a Polhemus FastSCAN Scorpion scanner. The weight of the participants can be recorded.

FIGS. 1A-2B show exemplary single frame foot images captured with a high speed camera. Video can be stepped frame by frame to identify heel contact. From this point, foot deformation can be measured by tracking marker displacement or by tracking the limits of the flattened tissue regions.

d. Data Reduction

The measurements from the pressure mat can be extracted using Research Foot software (version 6.7, Tekscan, Inc.). Data of heel and forefoot compression can be extracted by digitizing, using TEMA data-reduction software. The raw force and/or pressure data can be filtered to create an accurate output. Raw coordinate data from the high-speed video—representing deformation of the heel pad, metatarsal head, and toe—can be filtered using the Image Processing Toolbox in Matlab. The pressure transducer and/or force data can be synchronized with the time history of tissue deformation.

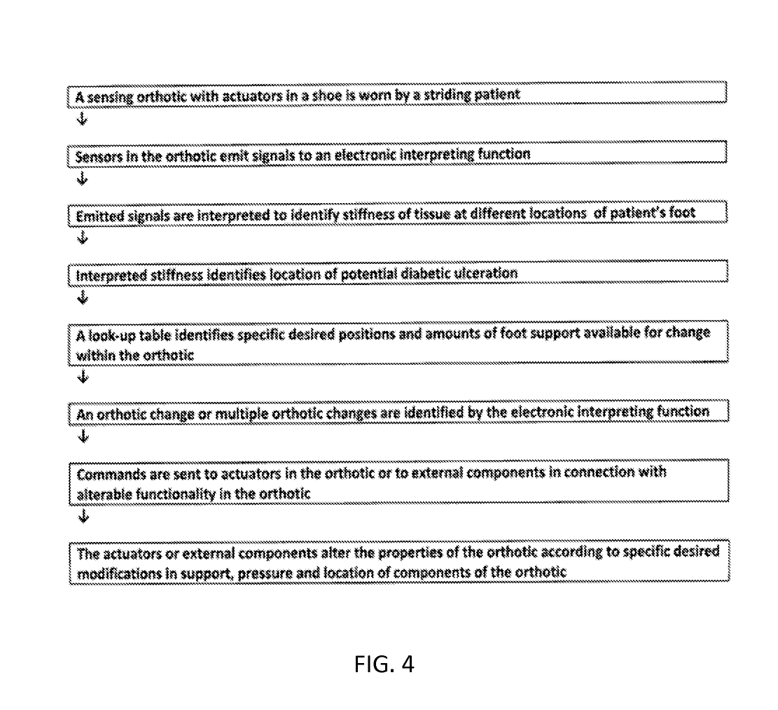

The System Identification Toolbox in Matlab can incorporate the pressure-tissue deformation data and/or force-tissue deformation data as an input in order to construct dynamic models of the various regions of the plantar surface during support. First, however, linear models can be tested. If these models do not yield an acceptable performance, nonlinear models can be used to improve the accuracy of the proposed models. A finite element model can be constructed for the regions of the foot to create material models of the tissue that match the experimental data. The pressure-tissue deformation model of the diabetic foot can inform the development of the smart orthotic (described below), which can incorporate smart materials and actuators to redistribute pressure on the foot, thus reducing the development of ulceration. A schematic of the flow of data collection and reduction is given in FIG. 4.

e. Statistical Analysis

The results of these two models (normal vs. diabetic foot) can be compared descriptively in order to identify the differences in stiffness of the various regions of the footpad between the test groups. This descriptive information can be used to support the validity of the diabetic foot models in that it appropriately represents compressive characteristics that are unique to the diabetic population.

The upper 95% confidence limits of the pressure measurement from the normal group can be obtained for each region of the footpad. The sensitivity of each region can then be calculated as the proportion of the patients in the diabetic foot group having higher pressure measurements. The confidence interval of the sensitivity can also be calculated. The sensitivity of the study can also be computed by combining the pressure measurements from all footpad regions to evaluate model differences.

### Orthotic Device

A system and method are provided for in situ sensitometric evaluation of an orthotic in footwear of a patient. By sensitometric it is meant that sensors are provided in contact with or embedded into the orthotic. Also associated with (in direct contact with a surface of the orthotic or embedded in the orthotic) are actuators. As used herein, the term “actuator” refers to elements that can impose physical movements, shifts, inflation, angular adjustments, internal pressure and other physical effects on the orthotic. As further disclosed herein, the sensors and actuators can be operatively connected with logic, processing, and/or signal activating technology (e.g., field programmable gated arrays or Application-Specific Integrated Circuits (ASICs) to thereby enable the in situ adjustment of the orthotic using the actuators associated with the orthotic in the footwear.

At least two different types of sensors can be distributed along various regions of the footpad of a patient (e.g., heel, metatarsal and toes). Pressure and displacement measurements (and thermal or temperature measurements) can also be taken at other locations, including, for example and without limitation, the sides of the feet (as opposed to only the bottom or footpad) and even the tops of the feet. In exemplary aspects, the at least two sensors can comprise pressure sensors to measure pressure applied to specific areas within the footpad (or other area) during the gait of a patient and displacement sensors which measure deformation in the footpad (or other area) in the various regions during application of gait pressure.

It is contemplated that the at least two sensors can comprise any commercially available sensors. In exemplary aspects, the at least two sensors can comprise at least one piezoelectric sensor. The sensors can be positioned at depths, orientations and positions within or on the orthotic so that specifically desired information is detected and provided by the sensor. In response to pressure, stress, elongation, and the like, the sensors can be configured to emit electrical signals that are transmitted to a processing or logic interpreting element. If transmitted to a processor, the signals can be received, source identified (and, optionally, time stamped), compared to a look-up table to interpret the quantitative (vector and/or amount) and qualitative (e.g., where located, interpretation of the propriety of the property or force measured) significance of the data created from the signals transmitted by the processor. The processor can then evaluate the data and, either automatically or upon user initiated activity effect a corresponding response by the actuators of the system.

Optionally, in exemplary aspects, the orthotic can comprise an inflatable component having multiple independent compartments (e.g., 2, 3, 4, 5, 6, 7, 8, 9, or 10), with each independent compartment being selectively inflatable. In these aspects, it is contemplated that each independent compartment can have a respective pressure sensor that is configured to produce an output indicative of the pressure created within the compartment as a result of the gait of the subject. It is further contemplated that the pressure sensors associated with the independent compartments of the inflatable component can be positioned in operative communication with a processor as further disclosed herein. In exemplary aspects, the processor can be configured to receive the output from the pressure sensors. In these aspects, when the pressure (as indicated by the output from a pressure sensor) is excessive or insufficient as a result of the positioning or movement of the foot during the gait of the subject, the processor can be configured to send a signal to the corresponding actuator(s) to individually and accordingly adjust pressure in the independent compartments where pressure adjustments are needed. In exemplary aspects, a single actuator (e.g., a pressure pump) can be used with separate controllable pathways to the individual compartments (e.g., a series of valves controlling directed flow through tubes to each respective independent chamber). Alternatively, in other exemplary aspects, multiple actuators can be associated with individual or subsets of the independent compartments. In exemplary aspects, respective independent compartments can be provided under the heel, metatarsal heads, and each toe.

In addition to inflatable/deflatable compartments, the orthotic can comprise compartments that can be filled with fluids, different types of fluids (e.g., gas, liquids, viscoelastics, Newtonian fluids, non-Newtonian fluids, etc.). In exemplary aspects, the actuators can be mechanical actuators (e.g., post actuators, plate actuators) that are configured to move solid elements within the orthotic to increase stiffness, provide structural support in specific directions (vertically, horizontally, or at an angle), and/or to shift portions of the orthotic (e.g., extend the length of the orthotic by pushing the end of the heel backwards, and the like).

In exemplary aspects, the orthotic can comprise at least two sensors associated with the orthotic. In these aspects, the at least two sensors can be configured to collective sense pressure and tissue displacement in a foot of a patient in contact with the at least two sensors. It is contemplated that each of the at least two sensors can be configured to emit respective signals to the processor. It is further contemplated that the processor can be configured to interpret the signals from the at least two sensors to thereby provide an indication of tissue stiffness based upon the signals from the at least two sensors. It is further contemplated that the processor can be configured to send commands to the actuators of the orthotic to alter physical properties of the orthotic to adjust one or more physical properties to thereby assist in medical treatment of the stiffness of tissues in the foot of the patient.

In exemplary aspects, the orthotic can be shaped to fit on the inside of a shoe and lie against a foot of a patient wearing the shoe. In these aspects, it is contemplated that the orthotic can comprise a flexible structural material. It is further contemplated that the disclosed active components (e.g., compartments, actuators) can be embedded within the flexible structural material of the orthotic to thereby permit adjustment of pressure and/or stiffness of various areas of the orthotic. It is still further contemplated that the disclosed active components can optionally be configured to adjust the position of one or more segments of the orthotic. In exemplary aspects, each active component can be operatively associated with at least one sensor as disclosed herein. In these aspects, the at least one sensor associated with each active component can be configured to sense at least one of pressure and tissue displacement in a foot of a patient. It is further contemplated that each sensor can be operatively coupled to the processor through a communication link as is known in the art, thereby permitting interpretation of the signals produced by each respective sensor. In exemplary aspects, the processor can be operatively coupled a return lead that is configured for operative communication with the active components (e.g., actuators as further disclosed herein).

In use, and with reference to FIG. 4, the parameters of the orthotic can be selectively adjusted in an in situ manner. For example, in one aspect, a patient wearing a shoe or boot having a sensing orthotic (such as those described above) therein can be examined. In exemplary aspects, and as further disclosed herein, the sensing orthotic can have a plurality of sensors that are distributed along the various regions of the footpad (heel, metatarsal, and toes). The at least two types of sensors can be appropriately distributed about and/or within the orthotic to ensure that information regarding selected regions of the foot is provided. In exemplary aspects, the at least two sensors can comprise pressure sensors configured to measure the pressure applied to specific areas within the foot pad during gait and displacement sensors configured to measure the deformation of the footpad tissues in the various regions of the footpad under the gait pressure. During movement of the patient wearing the shoe and orthotic, real-time measurements of at least pressure and displacement can be contemporaneously sensed to create corresponding electrical signals (outputs). The electrical signals (outputs) from the sensing elements can be transmitted to a processor (or logic interpreting element) where the signals are identified and evaluated (e.g., processed). The processor can be configured to convert the processed signals into a code to evaluate the stiffness of the tissues within various regions of the footpad. The code can use system identification techniques (predetermined, standardized) to evaluate the stiffness of the tissues. Based on continuous monitoring of the patient wearing the orthotic, calculating the stiffness of the various areas of the footpad during gait, and comparing these values with those stored in a look-up table, the code can predict the onset of diabetic ulceration(s) in the patient. Optionally, the look-up table can comprise historical information concerning the typical physical characteristics of the feet of patients having diabetic ulcerations. A processor (or logic, e.g., FPGA or ASIC) can be alerted by the code and, if necessary, send a signal to the treating physician or directly to the actuators in the orthotic and advise the wearer of the orthotic to seek proper treatment or to cause a specific formulated treatment through the actuator(s). In exemplary aspects, the processor can be configured to transmit at least one of an e-mail, a text message, or an online alert to share the alert with a physician or other selected recipients. Optionally, in these aspects, it is contemplated that the alert can be generated through a smart phone application that is operatively connected to the processor.

If the first code identifies a potential for ulceration in one or several areas, the processor can also use another code/signal (a second code) concurrently. The input to the second code can be the pressure and displacement data of the sensors and the stiffness models identified by the first code. The second code can control a plurality of actuators that are placed within the orthotic to calculate the desired amounts of pressure or stiffness that should be produced by the actuators to moderate the pressure of the ulcerated area(s). The processor can send signals to at least one actuator associated with the orthotic that can alter at least one property of position, pressure and displacement of the orthotic. The processor can provide at least one signal to the at least one actuator to effect alteration of at least one property of position, pressure and displacement of the orthotic to moderate any detected deviation. The second code can use the principles of automatic control to ensure the output of the actuators is stable and matches the multiple phases of the gait. It is contemplated that the steps and procedures can be repeated as necessary as further adjustments are determined to be necessary.

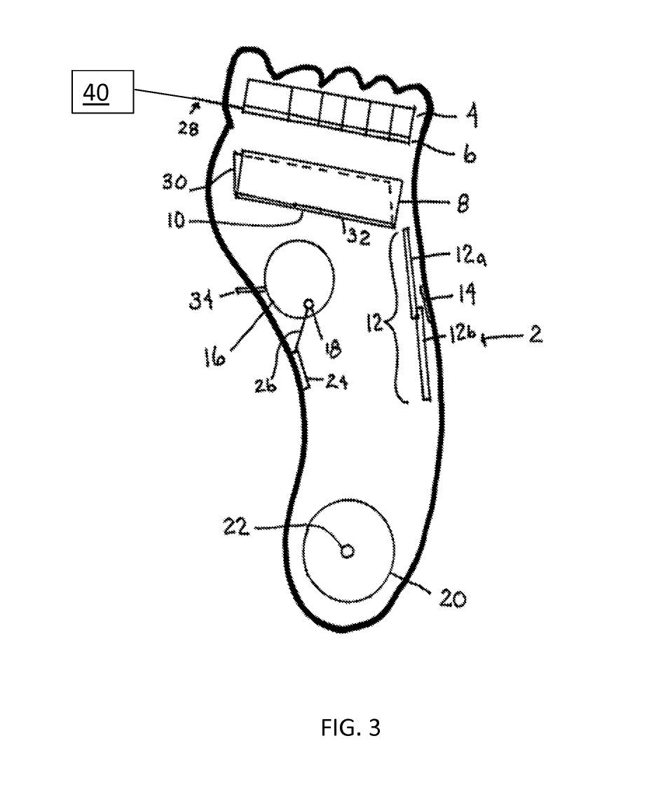

Reference to the Figures will further assist in appreciating the scope of the present technology. FIG. 3 shows a top view of an orthotic 2 for placement within a shoe, the orthotic having sensors 6, 10, 14, 18 and 22 and actuators 12 and 20 distributed therein. The orthotic can also have various compartments 4, 16, 20 spaced to interact with selected portions of the foot of the patient. The sensors 6 (six separate sensors are shown) are distributed alongside six separate controlled pressure compartments 4. The pressure in each of the six compartments 4 is separately controllable by allowing less or more fluid to be present within the compartments. Thus, in exemplary aspects, each compartment 4 can be positioned in fluid communication with a fluid source (not shown). An external lead 28 from the sensors 6 to a processor 40 is shown.

A single pressure controllable element 8 with a single sensor 10 with its own power supply is shown. The sensor 10 may also have a logic element contained therein, such as a field-programmable gated array (FPGA). Alternatively, the sensor 10 can be positioned in operative communication with a processor (e.g., processor 40). In exemplary aspects, a hinging motor element 32 can be operatively coupled with a backplate 30 that is configured for selective pivotal movement relative to a top surface of the orthotic to thereby provide desired support to the foot of the patient. In exemplary aspects, the hinging motor element 32 can be positioned in operative communication with a processor, and the processor can be configured to instruct the hinging motor element to relatively move backplate 30 in the controllable element 8 to adjust the height (and amount) of support provided by the element 8.

Also shown are two slideable plates 12a and 12b forming a side stiffening element 12. A sensor 14 can be used in conjunction with the side stiffening element 12. In exemplary aspects, the sensor 14 can be configured to measure stiffness. A motor or externally accessible gear system can be provided to relatively move the two plates 12a and 12b to thereby adjust the stiffness profile of the orthotic. In exemplary aspects, the sensor 14 and the two slideable plates 12a, 12b can be positioned in operative communication with a processor (e.g., processor 40) to permit monitoring and adjustment of stiffness as further disclosed herein.

An inflatable support element 16 is shown with a sensor 18 in communication through lead 26 to an external I/O port or wireless transmitter 24 which can be in further communication with a processor or logic element (e.g., processor 40). An externally extending tube 34 is shown through which fluid may be pumped to adjust pressure within the support element 16. Thus, it is contemplated that the tube 34 can provide fluid communication between a fluid source (not shown) and support element 16. In exemplary aspects, it is contemplated that the processor (e.g., processor 40) can be positioned in fluid communication with the fluid source to effect selective delivery or withdrawal of fluid from the support element 16.

A second inflatable element 20 is shown with a single integrated sensor, logic (FPGA) and fluid control component 22. However, it is contemplated that the integrated sensor of the element 20 can be positioned in operative communication with a processor (e.g., processor 40). As shown, the second inflatable element 20 can be configured for positioning proximate the heel of a patient. As variations in heel pressure are determined, signals can be interpreted by the logic or processor as requiring or not requiring increased/decreased pressure to be provided by the fluid control function of the component 20.

### Exemplary Aspects

In exemplary aspects, disclosed herein is a system for modeling tissue deformation of a foot of a patient as the patient walks relative to an axis of movement, the patient having a weight, the system comprising: means for producing at least one pressure output indicative of a pressure applied by the foot of the patient as the patient walks relative to the axis of movement; means for producing one or more images of the foot of the patient as the patient walks relative to the axis of movement; a processor configured to receive the at least one pressure output and the one or more images, wherein the processor is configured to determine deformation of the foot of the patient based upon the one or more images, and wherein the processor is further configured to correlate the at least one pressure output to a corresponding deformation of the tissue of the foot of the patient to thereby produce a tissue deformation profile.

In other exemplary aspects, the means for producing the at least one pressure output comprises at least one pressure sensor.

In other exemplary aspects, the means for producing one or more images comprises at least one camera.

In other exemplary aspects, the at least one camera comprises at least first and second cameras, wherein the first camera has an orientation axis positioned substantially perpendicular to the axis of movement, and wherein the second camera has an orientation axis positioned substantially parallel to the axis of movement.

In other exemplary aspects, the means for producing at least one pressure output comprises a pressure-sensitive device positioned such that the axis of movement passes through the pressure-sensitive device, the pressure-sensitive device having a transverse axis substantially perpendicular to the axis of movement and a contact surface, wherein the at least one pressure sensor is configured to produce at least one pressure output indicative of a pressure applied to the contact surface as the patient walks over the pressure-sensitive device relative to the axis of movement.

In other exemplary aspects, the pressure-sensitive device is a mat.

In other exemplary aspects, the pressure-sensitive device is an insole.

In other exemplary aspects, the orientation axis of the first camera is positioned substantially parallel to the transverse axis of the mat, and the orientation axis of the second camera is positioned substantially parallel to the axis of movement.

In other exemplary aspects, the processor is in operative communication with the at least one pressure sensor of the pressure sensitive device and the first and second cameras, wherein the processor is configured to receive the at least one pressure output from the at least one pressure sensor of the pressure sensitive device, and wherein the processor is configured to receive the one or more images produced by the first and second cameras.

In other exemplary aspects, the system further comprises a three-dimensional scanner configured to produce a three-dimensional image of the foot of the patient, wherein the processor is positioned in operative communication with the three-dimensional scanner and configured to receive the three-dimensional image of the foot of the patient.

In another exemplary aspect, disclosed herein is a method of producing an orthotic device, comprising: producing a tissue deformation profile using the tissue deformation modeling system; using the tissue deformation profile to select a desired configuration of the orthotic device; and producing the orthotic device in accordance with the desired configuration.

In other exemplary aspects, the orthotic device defines a top surface and comprises: at least two sensors; a processor positioned in operative communication with the at least two sensors, wherein the at least two sensors are configured to collectively sense pressure and tissue displacement in a foot of a patient in contact with the top surface of the orthotic device, and wherein each of the at least two sensors is configured to transmit a respective output to the processor.

In other exemplary aspects, the processor of the orthotic device is configured to determine the tissue stiffness of the foot of the patient based upon the outputs received from the at least two sensors.

In other exemplary aspects, the orthotic device comprises a flexible structural material, wherein the orthotic device defines a plurality of compartments, and wherein each compartment of the plurality of compartments is selectively collapsible and expandable to adjust pressure within the compartment.

In other exemplary aspects, the orthotic device further comprises a plurality of actuators, wherein each actuator is operatively coupled to at least one compartment of the orthotic device, and wherein the plurality of actuators are configured to selectively collapse and expand each respective compartment.

In other exemplary aspects, the processor is positioned in operative communication with the plurality of actuators, and the processor is configured to effect movement of the plurality of actuators sufficient to achieve desired physical parameters of each respective compartment.

In other exemplary aspects, the desired physical parameters of each respective compartment comprise at least one of pressure, stiffness, and position.

In other exemplary aspects, the processor of the orthotic device is in communication with a memory, and the memory stores a database comprising tissue stiffness parameters for patients having diabetic ulceration.

In other exemplary aspects, the processor of the orthotic device is configured to predict a likelihood of diabetic ulceration for the patient.

In other exemplary aspects, the processor of the orthotic device is configured to produce an output corresponding to a likelihood of diabetic ulceration.

In a further exemplary aspect, disclosed herein is a method of developing a tissue deformation profile for a foot of a patient, comprising: producing a plurality of pressure outputs indicative of the pressure applied by the foot during walking; producing one or more images depicting the compression of the foot during walking; determining, using a processor, the deformation of the foot based upon the one or more images depicting the compression of the foot; and correlating, using a processor, the pressure applied by the foot and the deformation of the foot to produce the tissue deformation profile.

Other variations will be understood by those skilled in the art when operating within the scope of technology described herein without extending beyond the generically intended scope of the invention.

### REFERENCES

1. Rowe V L, Kaufman J L, Talaver F. Diabetic ulcers. Medscape Web site. http://emedicine.medscape.com/article/460282-overview. Updated Sep. 25, 2012. Accessed Oct. 29, 2013.
2. Maluf K S, Mueller M J. Comparison of physical activity and cumulative plantar tissue stress among subjects with and without diabetes mellitus and a history of recurrent plantar ulcers. Clin Biomech. 2003; 18:567-575.
3. Mueller M J, Hastings M, Commean P K, et al. Forefoot structural predictors of plantar pressures during walking in people with diabetes and peripheral neuropathy. J Biomech. 2003; 36:1009-1017.
4. Gu Y, Li J, Ren X, Lake M J, Zeng Y. Heel skin stiffness effect on the hind foot biomechanics during heel strike. Skin Res Technol. 2010; 16:291-296.
5. Fontanella C G, Matteoli S, Carniel E L, et al. Investigation on the load-displacement curves of a human healthy heel pad: in vivo compression data compared to numerical results. Med Eng Phys. 2012; 34:1253-1259.
6. Chitikeshi V, Mahajan A, Chitikeshi S, Gupta R, Schoen M. An intelligent foot monitoring system for diabetic patients to prevent foot ulcerations. Paper presented at: ASME International Mechanical Engineering Congress and Exposition; Nov. 5-10, 2006; Orlando, Fla. http://proceedings.asmedigitalcollection.asme.org/proceeding.aspx?articleid=1603148
7. Atlas E, Yizhar Z, Gefen A. The diabetic foot load monitor: a portable device for real-time subject-specific measurements of deep plantar tissue stresses during gait. J Med Devices. 2008; 2(1).
8. Atlas E, Yizhar Z, Khamis S, Slomka N, Hayek S, Gefen A. Utilization of the foot load monitor for evaluating deep plantar tissue stresses in patients with diabetes: proof-of-concept studies. Gait Posture. 2009; 29:377-382.
9. World Health Organization. Burden: mortality, morbidity and risk factors. World Health Organization Web site. http://www.who.int/nmh/publications/ncd_report_chapter1.pdf. Accessed Oct. 29, 2013.
10. The Office of Minority Health. Diabetes and Hispanic Americans. U.S. Department of Health & Human Services Web site. http://minorityhealth.hhs.gov/templates/content.aspx?ID=3324. Updated Mar. 3, 2013. Accessed Oct. 29, 2013.
11. Cubit Planning, Inc. Nevada demographics summary. Cubit Web site. http://www.nevada-demographics.com/. Published 2013. Accessed Oct. 29, 2013.
12. Pepe M. Combining diagnostic test results to increase accuracy. Biostatistics (2000), 1, 2, pp. 123-140.
13. Ledoux W R, Shofer J B, Smith D G, Sullivan K, Hayes S G, et al. Relationship between foot type, foot deformity, and ulcer occurrence in the high-risk diabetic foot. J Rehab Res Devel. 2005:42:665-672.

1. A system for modeling tissue deformation of a foot of a patient as the patient walks relative to an axis of movement, the patient having a weight, the system comprising:
means for producing at least one pressure output indicative of a pressure applied by the foot of the patient as the patient walks relative to the axis of movement;
means for producing one or more images of the foot of the patient as the patient walks relative to the axis of movement;
a processor configured to receive the at least one pressure output and the one or more images,
wherein the processor is configured to determine deformation of the foot of the patient based upon the one or more images, and
wherein the processor is further configured to correlate the at least one pressure output to a corresponding deformation of the tissue of the foot of the patient to thereby produce a tissue deformation profile.

2. The system of claim 1, wherein the means for producing the at least one pressure output comprises at least one pressure sensor.

3. The system of claim 2, wherein the means for producing one or more images comprises at least one camera.

4. The system of claim 3, wherein the at least one camera comprises at least first and second cameras, wherein the first camera has an orientation axis positioned substantially perpendicular to the axis of movement, and wherein the second camera has an orientation axis positioned substantially parallel to the axis of movement.

5. The system of claim 4, wherein the means for producing at least one pressure output comprises a pressure-sensitive device positioned such that the axis of movement passes through the pressure-sensitive device, the pressure-sensitive device having a transverse axis substantially perpendicular to the axis of movement and a contact surface, wherein the at least one pressure sensor is configured to produce at least one pressure output indicative of a pressure applied to the contact surface as the patient walks over the pressure-sensitive device relative to the axis of movement.

6. The system of claim 5, wherein the pressure-sensitive device is a mat.

7. The system of claim 5, wherein the pressure-sensitive device is an insole.

8. The system of claim 5, wherein the orientation axis of the first camera is positioned substantially parallel to the transverse axis of the mat, and wherein the orientation axis of the second camera is positioned substantially parallel to the axis of movement.

9. The system of claim 8, wherein the processor in operative communication with the at least one pressure sensor of the pressure sensitive device and the first and second cameras, wherein the processor is configured to receive the at least one pressure output from the at least one pressure sensor of the pressure sensitive device, and wherein the processor is configured to receive the one or more images produced by the first and second cameras.

10. The system of claim 9, further comprising a three-dimensional scanner configured to produce a three-dimensional image of the foot of the patient, wherein the processor is positioned in operative communication with the three-dimensional scanner and configured to receive the three-dimensional image of the foot of the patient.

11. A method of developing a tissue deformation profile for a foot of a patient, comprising:
producing a plurality of pressure outputs indicative of the pressure applied by the foot during walking;
producing one or more images depicting the compression of the foot during walking;
determining, using a processor, the deformation of the foot based upon the one or more images depicting the compression of the foot; and
correlating, using a processor, the pressure applied by the foot and the deformation of the foot to produce the tissue deformation profile.

12. The method of claim 11, further comprising:
using the tissue deformation profile to select a desired configuration of an orthotic device; and
producing the orthotic device in accordance with the desired configuration.

13. The method of claim 12, wherein the orthotic device defines a top surface and comprises:
at least two sensors;
a processor positioned in operative communication with the at least two sensors,
wherein the at least two sensors are configured to collectively sense pressure and tissue displacement in a foot of a patient in contact with the top surface of the orthotic device, and
wherein each of the at least two sensors is configured to transmit a respective output to the processor.

14. The method of claim 13, wherein the processor of the orthotic device is configured to determine the tissue stiffness of the foot of the patient based upon the outputs received from the at least two sensors.

15. The method of claim 14, wherein the orthotic device comprises a flexible structural material, wherein the orthotic device defines a plurality of compartments, and wherein each compartment of the plurality of compartments is selectively collapsible and expandable to adjust pressure within the compartment.

16. The method of claim 15, wherein the orthotic device further comprises a plurality of actuators, wherein each actuator is operatively coupled to at least one compartment of the orthotic device, and wherein the plurality of actuators are configured to selectively collapse and expand each respective compartment.

17. The method of claim 16, wherein the processor is positioned in operative communication with the plurality of actuators, and wherein the processor is configured to effect movement of the plurality of actuators sufficient to achieve desired physical parameters of each respective compartment.

18. The method of claim 17, wherein the desired physical parameters of each respective compartment comprise at least one of pressure, stiffness, and position.

19. The method of claim 14, wherein the processor of the orthotic device is in communication with a memory, and wherein the memory stores a database comprising tissue stiffness parameters for patients having diabetic ulceration.

20. The method of claim 19, wherein the processor of the orthotic device is configured to predict a likelihood of diabetic ulceration for the patient.

21. The method of claim 20, wherein the processor of the orthotic device is configured to produce an output corresponding to a likelihood of diabetic ulceration.
